(19) United States
(12) Patent Application Publication
(10) Pub. No.: US 2022/0000746 A1
(43) Pub. Date: Jan. 6, 2022

(54) BIS(TRIETHOXYSILYLPROPYL)AMINE COMBINED WITH AN ALKANE

(71) Applicant: Henkel AG & Co. KGaA, Duesseldorf (DE)

(72) Inventors: RENE KROHN, Norderstedt (DE); ERIK SCHULZE ZUR WIESCHE, Bielefeld (DE)

(73) Assignee: Henkel AG & Co. KGaA, Duesseldorf (DE)

(21) Appl. No.: 17/290,722

(22) PCT Filed: Oct. 31, 2019
(86) PCT No.: PCT/EP2019/079783

§ 371 Date: Apr. 30, 2021

(51) Int. Cl. A61K 8/58; A61K 8/31; A61Q 5/00

(52) U.S. Cl. CPC A61K 8/585; A61K 2800/31; A61Q 5/002; A61K 8/31

(57) ABSTRACT

The present disclosure relates to an active ingredient composition for care and surface modification of human hair: The present disclosure especially relates to a cosmetic product for the treatment of a keratinous material, comprising 1) at least one organic silicon compound and b) at least one branched or linear C8 to C30 alkane, wherein the cosmetic product is suitable to nourish damaged hair.

### CROSS-REFERENCE TO RELATED APPLICATION

This application is a U.S. National-Stage entry under 35 U.S.C. § 371 based on International Application No. PCT/EP2019/079783, filed Oct. 31, 2019, which was published under PCT Article 21(2) and which claims priority to German Application No. 10 2018127190.5, filed Oct. 31, 2018, which are all hereby incorporated in their entirety by reference.

### TECHNICAL FIELD

The present disclosure relates to cosmetic products for the treatment of a keratinous material, wherein the product comprises an organic silicon compound and a branched or linear C8-C30 alkane, as well as the use of the cosmetic product.

### BACKGROUND

External stressing of the hair due to chemical materials from a variety of different sources poses challenges for the development of cosmetic care products. Air and water pollution adversely affect skin and hair. The most important air pollutants include polycyclic aromatic hydrocarbons, volatile organic compounds, nitrogen oxide (NOx), particles and cigarette smoke. The presence of other air pollutants and to UV radiation can reinforce the effect of various air pollutants.

It is known that the toxicity of gaseous pollutants of the air, such as sulfur dioxide, ozone and nitrogen oxides, especially depends on their initiator activity for free radicals, which causes damages in living beings. Free radicals are metabolic products that also occur naturally in the body. In large quantities, free radicals can pander to irritations and inflammations and accelerate the aging process. Such cases are referred to as oxidative damage. Free radicals can also cause hair damage, evinced, for example, by a reduction in the shine as well as of the grip and/or fading of the hair color.

Particles are a complete mixture that contains metals, minerals, organic toxins and/or biological material. They can also aid the formation of free radicals.

Furthermore, frequently changing customer requests regarding a certain finish of the hair are linked to a recurrent chemical stress on the hair. For example, hair dyes stress the hair, due to which, an intensive care may be necessary.

Organic compounds of silica from the group of silanes are described in the state of the art, which comprise at least one hydroxy group and/or hydrolysable group. Due to the presence of hydroxy groups and/or hydrolysable groups, silanes are reactive substances that hydrolyze or oligomerize or polymerize in the presence of water. When using on a keratinous material, oligomerization or polymerization of the silanes initiated by the presence of water eventually causes the formation of a film that can provide protection.

There is a requirement for a product that can heal the adverse effects of chemical hair treatment. In particular, there is a need for a post-treatment product that is capable of curing the damage to the hair surface.

### BRIEF SUMMARY

Cosmetic products for the treatment of a keratinous material are provided herein. In an embodiment, a cosmetic product includes
a) at least one organic silicon compound that comprises one to three silicon atoms and
b) at least one branched or linear alkane, wherein the alkane is a C8-C30 alkane.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The objective forming the basis of the present disclosure is in providing a product with an improved care and/or protective properties. In particular, the objective underlying the present disclosure was to provide a cosmetic product that, as a post-treatment product, heals any damage to the hair surface.

This objective is achieved by employing a cosmetic product for treating a keratinous material, comprising

a) at least one organic silicon compound that contains one to tree silicon atoms and
b) at least one branched or linear alkane, wherein the alkane is a C8-C30 alkane, preferably a C10-C24 alkane, more preferably a C12-C18 alkane and even more preferably a C14-C16 alkane.

Keratinous material includes hair, skin, nails (such as fingernails and/or toenails). Wool, furs and feathers also fall under the definition of keratinous material.

Preferably, a keratinous material is understood to be human hair, human skin and human nails, especially fingernails and toenails. Especially preferably, keratinous material is understood to be human hair in particular.

As the first ingredient essential for the invention, the cosmetic product for the treatment of a keratinous material contains at least one organic silicon compound that contains one to three silicon atoms. Preferred organic silicon compounds are selected from silanes with one, two or three silicon atoms, where the organic silicon compound comprises one or more hydroxyl groups and/or hydrolyzable groups per molecule.

Organic silicon compounds, alternatively called organosilicon compounds, are compounds which either have a direct silicon-carbon bond (Si—C) or in which the carbon is bonded to the silicon atom via an oxygen, nitrogen or sulfur atom. The organic silicon compounds are compounds containing one to three silicon atoms. Organic silicon compounds preferably contain one or two silicon atoms.

According to IUPAC rules, the term silane stands for a group of chemical compounds based on a silicon skeleton and hydrogen. In organic silanes, the hydrogen atoms are completely or partially replaced by organic groups such as (substituted) alkyl groups and/or alkoxy groups. In organic silanes, some of the hydrogen atoms may also be replaced by hydroxy groups.

Composition (a) contains at least one organic silicon compound selected from silanes having one, two or three silicon atoms, wherein the organic silicon compound comprises one or more hydroxyl groups or hydrolyzable groups per molecule.

As part of a particularly preferred embodiment, the product for the treatment of a keratinous material features at least one organic silicon compound, selected from silanes having one, two or three silicon atoms, the organic silicon compound further comprising one or more basic groups and one or more hydroxyl groups or hydrolyzable groups per molecule.

This basic group can be, for example, an amino group, an alkylamino group or a dialkylamino group, which is preferably connected to a silicon atom via a linker. The basic group is preferably an amino group, a C₁-C₆ alkylamino group or a Di(C₁-C₆) alkylamino group.

The hydrolyzable group(s) is (are) preferably a C₁-C₆ alkoxy group, especially an ethoxy group or a methoxy group. It is preferred when the hydrolyzable group is directly bonded to the silicon atom. For example, if the hydrolyzable group is an ethoxy group, the organic silicon compound preferably contains a structural unit R′R″R″′Si—O—CH₂—CH₃. The residues R′, R″ and R′″ represent the three remaining free valences of the silicon atom.

Particularly good results could be obtained if the product for the treatment of a keratinous material contains at least one organic silicon compound of formula (I) and/or (II).

The compounds of formulas (I) and (II) are organic silicon compounds selected from silanes having one, two or three silicon atoms, the organic silicon compound comprising one or more hydroxyl groups and/or hydrolysable groups per molecule.

In another specifically preferred embodiment, the product for the treatment of a keratinous material contains at least one organic silicon compound of formula (I) and/or (II).

R₁R₂N-L-Si(OR₃)ₐ(R₄)_{b} (I),
where

R₁, R₂ both represent a hydrogen atom, and

L represents a linear, double-bonded C₁-C₆-alkylene group, preferably a propylene group (—CH₂—CH₂—CH₂—) or an ethylene group (—CH₂—CH₂—),

R₃, R₄ independently represent a methyl group or an ethyl group,

a stands for the number 3 and
b stands for the number 0,

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
where

R5, R5′, R5″ independently represent a hydrogen atom or a C₁-C₆ alkyl group,

R6, R6′ and R6″ independently represent a C₁-C₆ alkyl group,

A, A′, A″, A′″ and A″″ independently represent a linear or branched double-bonded C₁-C₂₀ alkylene group,

R₇ and R₈ independently represent a hydrogen atom, a C₁-C₆ alkyl group, a hydroxy C₁-C₆ alkyl group, a C₂-C₆ alkenyl group, an amino C₁-C₆ alkyl group or a group of formula (III)

-(A″″)-Si(R₆″)_{d}″(OR₅″)_{c}″ (III),
c, stands for an integer from 1 to 3,
d stands for the integer 3-c,
c′ stands for an integer from 1 to 3,
d′ stands for the integer 3-c′,
c″ stands for an integer from 1 to 3,
d″ stands for the integer 3-c″,
e stands for 0 or 1,
f stands for 0 or 1,
g stands for 0 or 1,
h stands for 0 or 1,
provided that at least one of e, f, g and h is different from 0.

The substituents R₁, R₂, R₃, R₄, R₅, R₅′, R₅″, R₆, R₆′, R₆″, R₇, R₈, L, A, A′, A″, A′″ and A″″ in the compounds of formula (I) and (II) are explained below as examples: Examples of a C₁-C₆ alkyl group are the groups methyl, ethyl, propyl, isopropyl, n-butyl, s-butyl and t-butyl, n-pentyl and n-hexyl. Propyl, ethyl and methyl are preferred alkyl radicals. Examples of a C₂-C₆ alkenyl group are vinyl, allyl, but-2-enyl, but-3-enyl and isobutenyl, preferred C₂-C₆ alkenyl radicals are vinyl and allyl. Preferred examples of a hydroxy C₁-C₆ alkyl group are a hydroxymethyl, a 2-hydroxyethyl, a 2-hydroxypropyl, a 3-hydroxypropyl, a 4-hydroxybutyl group, a 5-hydroxypentyl and a 6-hydroxyhexyl group; a 2-hydroxyethyl group is particularly preferred. Examples of an amino C₁-C₆ alkyl group are the aminomethyl group, the 2-aminoethyl group, the 3-aminopropyl group. The 2-aminoethyl group is particularly preferred. Examples of a linear double-bonded C₁-C₂₀-alkylene group include for example, the methylene group (—CH₂—), the ethylene group (—CH₂—CH₂—), the propylene group (—CH₂—CH₂—CH₂—) and the butylene group (—CH₂—CH₂—CH₂—CH₂—). The propylene group (—CH₂—CH₂—CH₂—) is particularly preferred. From a chain length of 3 C atoms, double-bonded alkylene groups can also be branched. Examples of branched double-bonded C₃-C₂₀ alkylene groups are (—CH₂—CH(CH₃)—) and (—CH₂—CH(CH₃)—CH₂—).

In the organic silicon compounds of the formula (I)

R₁R₂N-L-Si(OR₃)ₐ(R₄)_{b} (I),

the radicals R₁ and R₂ independently of one another represent a hydrogen atom or a C₁-C₆ alkyl group. In particular, the radicals R₁ and R₂ both represent a hydrogen atom.

In the middle part of the organic silicon compound is the structural unit or the linker -L- which stands for a linear or branched, double-bonded C₁-C₂₀ alkylene group.

Preferably -L- stands for a linear, double-bonded C₁-C₂₀ alkylene group. Further preferably -L- stands for a linear double-bonded C₁-C₆ alkylene group. Particularly preferred -L stands for a methylene group (—CH₂—), an ethylene group (—CH₂—CH₂—), propylene group (—CH₂—CH₂—CH₂—) or butylene (—CH₂—CH₂—CH₂—CH₂—). In particular, L stands for a propylene group (—CH₂—CH₂—CH₂—)

The organic silicon compounds of formula (I)

R₁R₂N-L-Si(OR₃)ₐ(R₄)_{b} (I),

carry the silicon-containing grouping —Si(OR₃)ₐ(R₄)_{b} at one end.

In the terminal structural unit —Si(OR₃)ₐ(R₄)_{b}, R₃ is hydrogen or C₁-C₆ alkyl group, and R₄ is C₁-C₆ alkyl group. Particularly preferred, R₃ and R₄ independently of each other represent a methyl group or an ethyl group.

Here a stands for an integer from 1 to 3, and b stands for the integer 3-a. If a stands for the number 3, then b is equal to 0. If a stands for the number 2, then b is equal to 1. If a stands for the number 1, then b is equal to 2.

The best protection from the negative effects of water and/or air pollutants (“Anti-pollution” effect) and the best care of stressed hair could be obtained, if the product for the treatment of a keratinous material contains at least one organic silicon compound of the formula (I) or formula (II), in which the radicals R₃, R₄ independently represent a methyl group or an ethyl group.

Especially well-suited organic silicon compounds of the formula (I) are:

### (3-Aminopropyl)triethoxysilane

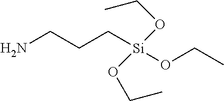

### (3-Aminopropyl)trimethoxysilane

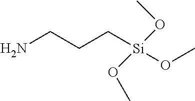

### 1-(3-Aminopropyl)silantriol

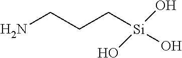

### (2-Aminoethyl)triethoxysilane

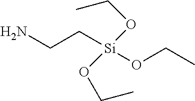

### (2-Aminoethyl)trimethoxysilane

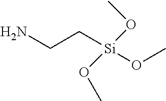

### 1-(2-Aminoethyl)silantriol

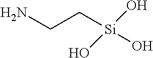

### (3-Dimethylaminopropyl)triethoxysilane

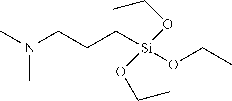 [SMILES: CCO[Si](CCCN(C)C)(OCC)OCC]

### (3-Dimethylaminopropyl)trimethoxysilane

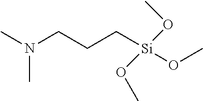

### 1-(3-Dimethylaminopropyl)silantriol

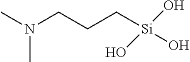

### (2-Dimethylaminoethyl)triethoxysilane

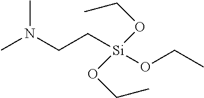

### (2-Dimethylaminoethyl)trimethoxysilane and/or

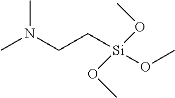

### 1-(2-Dimethylaminoethyl)silantriol

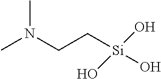

The aforementioned organic silicon compound of formula (I) is commercially available.

(3-aminopropyl)trimethoxysilane, for example, can be purchased from Sigma-Aldrich. (3-aminopropyl)triethoxysilane is also commercially available from Sigma-Aldrich.

In a further embodiment, the product for the treatment of a keratinous material contains at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II).

The organosilicon compounds of formula (II) each carry the silicon-containing groups (R₅O)_{c}(R₆)_{d}Si— and —Si(R₆′)_{d′}(OR₅′)_{c} at both ends.

In the central part of the molecule of formula (II) there are the groups -(A)ₑ- and —[NR₇-(A′)]_{f}— and —[O-(A″)]_{g}- and —[NR₈-(A′″)]ₕ-. Here, each of the radicals e, f, g and h can independently of one another stand for the number 0 or 1, with the proviso that at least one of the radicals e, f, g and h is different from 0. In other words, an organic silicon compound of formula (II) contains at least one grouping from the group of -(A)- and —[NR₇-(A′)]- and —[O-(A″)]- and —[NR₈-(A′″)]-.

In the two terminal structural units (R₅O)_{c}(R₆)_{d}Sii- and —Si(R₆′)_{d′}(OR₅′)_{c}, the radicals R5, R5′, R5″ independently of one another represent a hydrogen atom or a C₁-C₆ alkyl group. The radicals R6, R6′ and R6″ independently represent a C₁-C₆ alkyl group.

Here a stands for an integer from 1 to 3, and d stands for the integer 3-c. If c stands for the number 3, then d is equal to 0. If c stands for the number 2, then d is equal to 1. If c stands for the number 1, then d is equal to 2.

Analogously c′ stands for a whole number from 1 to 3, and d′ stands for the whole number 3-c′. If c′ stands for the number 3, then d′ is 0. If c′ stands for the number 2, then d′ is 1. If c′ stands for the number 1, then d′ is 2.

A very high anti-pollution effect of the product for the treatment of a keratinous material can be obtained if the radicals c and c′ both stand for the number 3. In this case d and d′ both stand for the number 0.

In a further preferred embodiment, the product for the treatment of a keratinous material contains at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where

R5 and R5′ independently represent a methyl group or an ethyl group,

c and c′ both stand for the number 3 and

d and d′ both stand for the number 0.

If c and c′ are both the number 3 and d and d′ are both the number 0, the organic silicon compound of the present disclosure corresponds to formula (IIa)

(R₅O)₃Si-(A)ₑ-[NR₇-(A′)]_{f}—[O-(A″)]_{g}—[NR₈-(A′″)]ₕ—Si(OR₅′)₃ (IIa).

The radicals e, f, g and h can independently stand for the number 0 or 1, whereby at least one radical from e, f, g and h is different from zero. The abbreviations e, f, g and h thus define which of the groupings -(A)ₑ- and -[NR7-(A′)]_{f}- and —[O-(A″)]_{g}- and -[NR8-(A′″)]ₕ- are located in the middle part of the organic silicon compound of formula (II).

In this context, the presence of certain groupings has proved to be particularly beneficial in terms of increasing washability. Particularly good results were obtained when at least two of the residues e, f, g and h stand for the number 1. Especially preferred e and f both stand for the number 1. Furthermore, g and h both stand for the number 0.

If e and f both stand for the number 1 and g and h both stand for the number 0, the organic silicon compounds correspond to formula (IIb)

(R₅O)_{c}(R₆)_{d}Si-(A)-[NR₇-(A′)]—Si(R₆′)_{d′}(OR₅′)_{c′} (IIb).

The radicals A, A′, A″, A′″ and A″″ independently represent a linear or branched double-bonded C₁-C₂₀ alkylene group. Preferably the radicals A, A′, A″, A′″ and A″″ independently of one another represent a linear, double bond C₁-C₂₀ alkylene group. Further preferably the radicals A, A′, A″, A′″ and A″″ independently represent a linear double bond C₁-C₆ alkylene group. In particular, the radicals A, A′, A″, A′″ and A″″ independently of one another represent a methylene group (—CH₂—), an ethylene group (—CH₂—CH₂—), a propylene group (—CH₂—CH₂—CH₂—) or a butylene group (—CH₂—CH₂—CH₂—CH₂—). In particular, the residues A, A′, A″, A′″ and A″″ stand for a propylene group (—CH₂—CH₂—CH₂—).

If the radical f represents the number 1, then the organic silicon compound of formula (II) contains a structural grouping —[NR₇-(A′)]-.

If the radical h represents the number 1, then the organic silicon compound of formula (II) contains a structural grouping —[NR₈-(A′″)]-.

Herein, the radicals R₇ and R₈ independently represent a hydrogen atom, a C₁-C₆ alkyl group, a hydroxy C₁-C₆-alkyl group, a C₂-C₆ alkenyl group, an amino-C₁-C₆-alkyl group or a grouping of the formula (III)

-(A″″)-Si(R₆″)_{d}″(OR₅″)_{c}″ (III).

Very preferably, R₇ and R₈ independently represent a hydrogen atom, a methyl group, a 2-hydroxyethyl group, a 2-alkenyl group, a 2-aminoethyl group or a group of formula (III).

If the radical f represents the number 1 and the radical h represents the number 0, the organic silicon compound contains the grouping [NR₇-(A′)] but not the grouping —[NR₈-(A′″)] If the radical R7 now stands for a grouping of the formula (III), the product for the treatment of a keratinous material contains an organic silicone compound with 3 reactive silane groups.

In a further preferred embodiment, the product for the treatment of a keratinous material contains at least one organic silicon compound of formula (II)

(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),

where

e and f both stand for the number 1,

g and h both stand for the number 0,

A and A′ independently stand for a linear, double bond C₁-C₆-alkylene group

and

R7 represents a hydrogen atom, a methyl group, a 2-hydroxyethyl group, a 2-alkenyl group, a 2-aminoethyl group or a group of formula (III).

In a further preferred embodiment, the product for the treatment of a keratinous material contains at least one organic silicon compound of formula (II), wherein

e and f both stand for the number 1,

g and h both stand for the number 0,

A and A′ independently of one another represent a methylene group (—CH₂—), an ethylene group (—CH₂—CH₂—) or a propylene group (—CH₂—CH₂—CH₂),

and

R₇ represents a hydrogen atom, a methyl group, a 2-hydroxyethyl group, a 2-alkenyl group, a 2-aminoethyl group or a group of formula (III).

Organic silicon compounds of the formula (II) which are well suited for solving the problem are

### 3-(trimethoxysilyl)-N-[3-(trimethoxysilyl)propyl]-1-propanamine

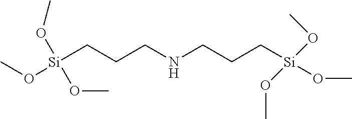

### 3-(Triethoxysilyl)-N-[3-(triethoxysilyl)propyl]-1-propanamine

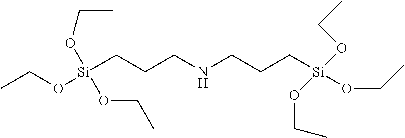 [SMILES: CCO[Si](CCCNCCC[Si](OCC)(OCC)OCC)(OCC)OCC]

### N-methyl-3-(trimethoxysilyl)-N-[3-(trimethoxysilyl)propyl]-1-propanamine

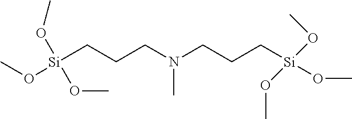

### N-Methyl-3-(triethoxysilyl)-N-[3-(triethoxysilyl)propyl]-1-propanamine

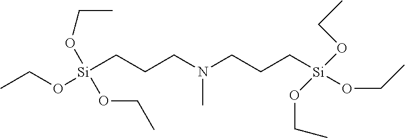 [SMILES: CCO[Si](CCCN(C)CCC[Si](OCC)(OCC)OCC)(OCC)OCC]

### 2-[Bis[3-(trimethoxysilyl)propyl]amino]-ethanol

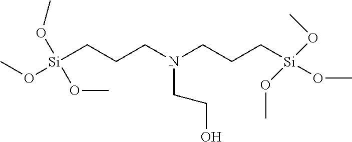

### 2-[bis[3-(triethoxysilyl)propyl]amino]ethanol

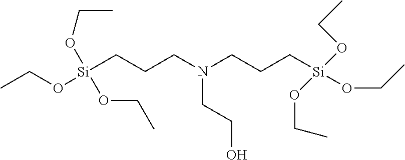 [SMILES: CCO[Si](CCCN(CCO)CCC[Si](OCC)(OCC)OCC)(OCC)OCC]

### 3-(Trimethoxysilyl)-N,N-bis[3-(trimethoxysilyl)propyl]-1-propanamine

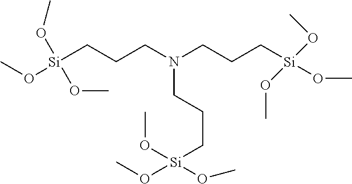

### 3-(Triethoxysilyl)-N,N-bis[3-(triethoxysilyl)propyl]-1-propanamine

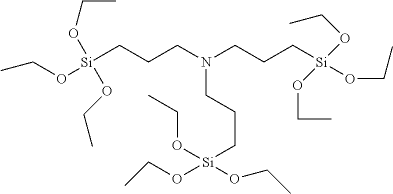 [SMILES: CCO[Si](CCCN(CCC[Si](OCC)(OCC)OCC)CCC[Si](OCC)(OCC)OCC)(OCC)OCC]

### N1,N1-Bis[3-(trimethoxysilyl)propyl]-1,2-ethanediamine

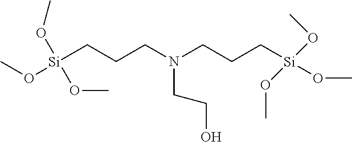

### N1,N1-Bis[3-(triethoxysilyl)propyl]-1,2-ethanediamine

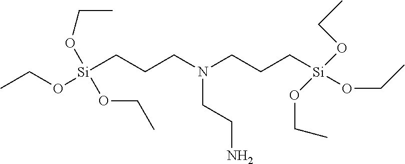 [SMILES: CCO[Si](CCCN(CCN)CCC[Si](OCC)(OCC)OCC)(OCC)OCC]

### N,N-Bis[3-(trimethoxysilyl)propyl]-2-propene-1-amine

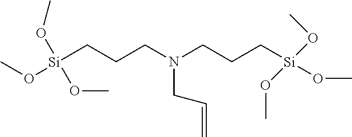

### N,N-Bis[3-(triethoxysilyl)propyl]-2-propene-1-amine

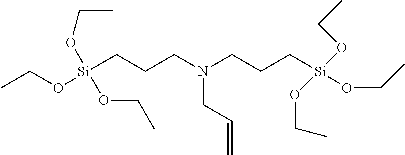 [SMILES: C=CCN(CCC[Si](OCC)(OCC)OCC)CCC[Si](OCC)(OCC)OCC]

The aforementioned organic silicon compounds of formula (II) are commercially available.

Bis(trimethoxysilylpropyl)amine with the CAS number 82985-35-1 can be purchased from Sigma-Aldrich.

Bis[3-(triethoxysilyl)propyl]amine, also designated as 3-(Triethoxysilyl)-N-[3-(triethoxysilyl)propyl]-1-propanamine, with the CAS number 13497-18-2 is, for example, commercially available from Sigma-Aldrich or can be commercially obtained from Evonik under the product name Dynasylan 1122.

N-methyl-3-(trimethoxysilyl)-N-[3-(trimethoxysilyl)propyl]-1-propanamine is alternatively referred to as bis(3-trimethoxysilylpropyl)-N-methylamine and can be purchased commercially from Sigma-Aldrich or Fluorochem.

3-(triethoxysilyl)-N,N-bis[3-(triethoxysilyl)propyl]-1-propanamine with the CAS number 18784-74-2 can be purchased for example from Fluorochem or Sigma-Aldrich.

It has also proved beneficial if the product for the treatment of a keratinous material contains at least one organic silicon compound of the formula (IV)

R₉Si(OR₁₀)ₖ(R₁₁)ₘ (IV).

The compounds of formula (IV) are organic silicon compounds selected from silanes having one, two or three silicon atoms, the organic silicon compound comprising one or more hydroxyl groups and/or hydrolysable groups per molecule.

The organic silicon compound(s) of formula (IV) may also be called a silane of the alkyl-alkoxy-silane or alkyl-hydroxy-silane type,

R₉Si(OR₁₀)ₖ(R₁₁)ₘ (IV),

where

R₉ represents a C₁-C₁₂, alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further preferred embodiment, the product for the treatment of a keratinous material contains, in addition to the organic silicon compound(s) of formula (I), at least one more organic silicon compound of formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a similarly preferred embodiment, the product for the treatment of a keratinous material contains, in addition to the organic silicon compound(s) of formula (II), at least one more organic silicon compound of formula (IV)

R₉Si(OR₁₀)ₖ(R₁₁)ₘ (IV),

where

R₉ represents a C₁-C₁₂ alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In a further preferred embodiment, the product for the treatment of a keratinous material contains, in addition to the organic silicon compound(s) of formula (I) and (II), at least one more organic silicon compound of formula (IV)

R₉Si(OR₁₀)ₖ(R₁₁)ₘ (IV),

where

R₉ represents a C₁-C₁₂, alkyl group,

R₁₀ represents a hydrogen atom or a C₁-C₆ alkyl group,

R₁₁ represents a C₁-C₆ alkyl group

k is an integer from 1 to 3, and

m stands for the integer 3-k.

In the organic silicon compounds of formula (IV), the radical R₉ represents a C₁-C₁₂ alkyl group. This C₁-C₁₂, alkyl group is saturated and can be linear or branched. Preferably R9 stands for a linear C₁-C₈ alkyl group. Preferably R₉ stands for a methyl group, an ethyl group, an n-propyl group, an n-butyl group, an n-pentyl group, an n-hexyl group, an n-octyl group or an n-dodecyl group. Particularly preferred, R₉ stands for a methyl group, an ethyl group or an n-octyl group.

In the organic silicon compounds of formula (IV), the radical R₁₀ represents a hydrogen atom or a C₁-C₆ alkyl group. Especially preferably, R₁₀ stands for a methyl group or an ethyl group.

In the organic silicon compounds of formula (IV), the radical R₁₁ represents a C₁-C₆ alkyl group. Especially preferably, R₁₁ stands for a methyl group or an ethyl group.

Furthermore k stands for a whole number from 1 to 3, and m stands for the whole number 3-k. If k stands for the number 3, then m is equal to 0. If k stands for the number 2, then m is equal to 1. If k stands for the number 1, then m is equal to 2.

A very high anti-pollution effect can be achieved if the product for the treatment of a keratinous material contains at least one organic silicon compound of the formula (IV) in which the radical k stands for the number 3. In this case the rest m stands for the number 0.

Organic silicon compounds of the formula (IV) which are well suited for solving the problem are

### Methyltrimethoxysilane

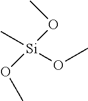

### Methyltriethoxysilane

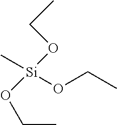

### Ethyltrimethoxysilane

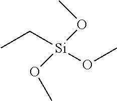

### Ethyltriethoxysilane

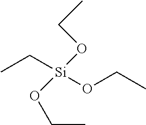

### n-Hexyltrimethoxysilane

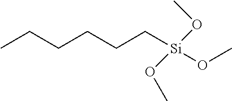

### n-Hexyltriethoxysilane

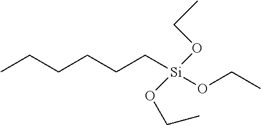 [SMILES: CCCCCC[Si](OCC)(OCC)OCC]

### n-Octyltrimethoxysilane

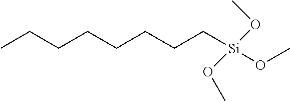

### n-Octyltriethoxysilane

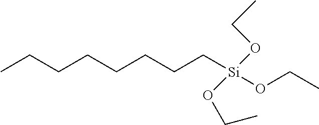 [SMILES: CCCCCCCC[Si](OCC)(OCC)OCC]

### n-Dodecyltrimethoxysilane and/or

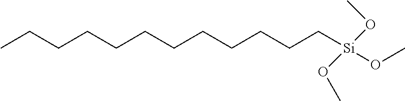 [SMILES: CCCCCCCCCCCC[Si](OC)(OC)OC]

### n-Dodecyltriethoxysilane

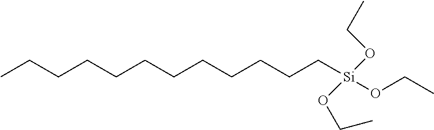 [SMILES: CCCCCCCCCCCC[Si](OCC)(OCC)OCC]

as well as propyltrimethoxysilane, propyltriethoxysilane, octadecyltrimethoxysilane and/or octadecyltriethoxysilane.

The organic silicon compounds described above are reactive compounds.

In this context, it has proved to be specifically preferable, if the product contains (3-Aminopropyl)triethoxysilane, i.e., an aminopropyl triethoxysilane (AMEO), and/or 3-(Triethoxysilyl)-N-[3-(triethoxysilyl)propyl]-1-propane amine, i.e., a Bis(triethoxysilylpropyl)amine, as organic silicon compound.

According to the preferred embodiment of the present invention, the organic silicon compound of the formula (I), particularly the (3-Aminopropyl)triethoxysilane, in a quantity of about 0.01 to about 10% by weight, preferably from about 0.02 to about 8% by weight, more preferably from about 0.05 to about 6% by weight, most preferably from about 0.1 to about 4% by weight, based on the total weight of the cosmetic product, contained in the cosmetic product, and/or the organic silicon compound of the formula (II), especially the 3-(Triethoxysilyl)-N-[3-(triethoxysilyl)propyl]-1-propane amine, is contained in a quantity of about 0.01 to about 10% by weight, preferably from about 0.02 to about 9% by weight, more preferably from about 0.05 to about 8% by weight, most preferably from about 0.1 to about 7% by weight, based on the total weight of the cosmetic product.

It has been found that it is possible to maintain particularly sturdy and uniform films on the keratinous material even if the product contains two structurally different organic silicon compounds, each of which contains one to three silicon atoms.

In a further preferred embodiment, product is exemplified in that the it contains at least one organic silicon compound of formula (I) and at least one organic silicon compound of formula (IV).

In an explicitly specifically preferred embodiment, a product is exemplified in that it contains at least one organic silicon compound of the formula (I), which is selected from the group of (3-Aminopropyl)triethoxysilane and (3-Aminopropyl)trimethoxysilane, and additionally contains at least one organic silicon compound of the formula (IV), which is selected from the group of methyltrimethoxysilane, methyltriethoxysilane, ethyltrimethoxysilane, ethyltriethoxysilane, propyltrimethoxysilane, propyltriethoxysilane, hexyltrimethoxysilane and hexyltriethoxysilane.

In a further preferred embodiment, a product is exemplified in that the product—based on the total weight of the product—contains:
From about 0.5 to about 5% by weight of at least one first organic silicon compound selected from the group of (3-aminopropyl)trimethoxysilane, (3-aminopropyl)triethoxysilane, (2-aminoethyl)trimethoxysilane, (2-aminoethyl)triethoxysilane, (3-dimethylaminopropyl)trimethoxysilane, (3-dimethylaminopropyl)triethoxysilane (2-dimethylaminoethyl)trimethoxysilane and (2-dimethylaminoethyl)triethoxysilane, and
From about 3.2 to about 10.0% by weight of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

In the case of organic silicon components with at least one hydrolysable group, the addition of small quantities of water already leads to hydrolysis. The hydrolysis products and/or organic silicon compounds with at least one hydroxy group can react with each other in a condensation reaction. For this reason, the product can contain organosilicon compounds with at least one hydrolysable group as well as their hydrolysis and/or condensation products. When using organosilicon compounds with at least one hydroxyl group, the product can contain organic silicon compounds with at least one hydroxyl group as well as their condensation products.

A condensation product is understood to mean a product that is created due to the reaction of at least two organic silicon compounds each with at least one hydroxyl group or hydrolysable group per molecule on splitting of water and/or splitting of an alkanol. The condensation products can, for example, be dimers, or even trimers or oligomers, where in the condensation products are always in balance with the monomers. Depending on the water quantity added or consumed in the hydrolysis, the balance shifts from monomeric organic silicon compounds to condensation product.

In the context of the present invention, specifications in weight-%—unless otherwise specified—always in relation to the total weight of the cosmetic product.

As the second ingredient essential to the invention, the cosmetic product for the treatment of a keratinous material contains at least one branched or linear alkane. During the work leading to this invention, it was found that it is especially advantageous for achieving a particularly good care effect, if the organic silicon compounds, such as 3-(Triethoxysilyl)-N-[3-(triethoxysilyl)propyl]-1-propanamine or (3-Aminopropyl)triethoxysilane, were combined with a branched or linear C8-C30 alkane. It was surprising to find that the combination of (3-aminopropyl)triethoxysilane with C8-C30 alkane raises the cosmetic acceptance. The hair is soft, the combability rises distinctly and the hair surface is, particularly with chemically treated hair, more hydrophobic.

According to preferred embodiments of the present invention, the alkane is a C10-C24 alkane, more preferably a C12-C18 alkane and even more preferably C14-C16 alkane.

The combination of the organosilicon compound, such as (3-aminopropyl)triethoxysilane and/or bis(triethoxysilylpropyl)amine, with which the alkane forms a layer on the hair. The hair surface is again hydrophobized—especially in the case of oxidation damage was due to oxidation—which leads to reduction in fizz. This also improves the combability.

According to a preferred embodiment, the cosmetic product further contains a skin moisturizer or other care agent that is selected from the group of glycerin, urea, hyaluronic acid, silanol ester of the hyaluronic acid, panthenol, taurine, ceramide, phytosterole, aloe vera extracts, creatine, creatinine, sodium hyaluronate, polysaccharides, biosaccharides gum-1, cucumber extracts, butylene glycol, propylene glycol, methyl propane diol, ethylhexylglycerin, sorbitol, amino acids, wherein glycine, glycine soy, histidine, tyrosine or tryptophan are especially preferred amino acids, amino acid derivatives, natural betaine compounds, pyrrolidone carboxylic acid or a salt of pyrrolidone carboxylic acid, lactic acid, lactates, especially sodium lactate, and/or ethylhexyloxyglycerin. Particularly, the selection of these skin moisturizers enhances the care character of the cosmetic product.

According to a preferred embodiment of the present invention, the quantity of branched or linear alkane in the cosmetic product is about 0.1 to about 30% by weight, preferably from about 0.5 to about 25% by weight more preferably from about 1 to about 20% by weight, even more preferably from about 2 to about 15% by weight based on the total weight of the cosmetic product.

According to a more preferred embodiment, the pH value of the cosmetic product lies in the range from about 1 to about 9, preferably from about 1.5 to about 8, more preferred from about 2 to about 7, even more preferably from about 2.5 to about 6 and the most preferably from about 3 to about 5.

According to another more preferred embodiment, the cosmetic product contains one or more surfactants. It is particularly preferred that the cosmetic product contain two structurally mutually different surfactants, wherein preferably the cosmetic product contains two structurally mutually different cationic surfactants, two structurally mutually different anionic surfactants, one cationic surfactant and a non-ionic surfactant, or one anionic surfactant and a non-ionic surfactant.

According to a preferred embodiment of the present invention, the cationic surfactant comprises a hydrophobic head group with a cationic charge and one or two hydrophobic end parts, wherein the hydrophobic end part or the hydrophobic end parts represent straight chained or branched, saturated or mono- or poly-unsaturated alkyl groups, which preferably feature a chain length of C6 to C30, preferably C8 to C26, especially preferred C10 to C22. According to a further preferred embodiment, the cationic surfactant features an ester function, an ether function, a ketone function, an alcohol function or an amide function.

According to other preferred embodiments of the present invention, the cosmetic product contains at least one cationic surfactant of the formula (V),

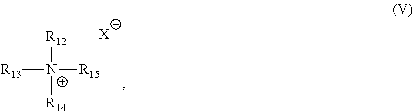

wherein
R₁₂, R₁₃, R₁₄ independently represent an C1-C6-Alkyl group, a C2-C6-alkenyl group or a C2-C6-hydroxyalkyl group,
R₁₅ represents a C8-C28-alkyl group, preferably a C10-C22-alkyl group and
X— stands for a physiologically compatible anion,
and/or the cosmetic product contains at least one cationic surfactant of the formula (VI),

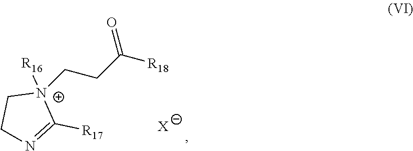

wherein
R₁₆ represents a C1-C6 alkyl group
R₁₇, R₁₈ independently represent a C7-C27-alkyl group, preferably an C10-C22-alkyl group and
X— stands for a physiologically compatible anion,
and/or the cosmetic product contains at least one cationic surfactant of the formula (VII),

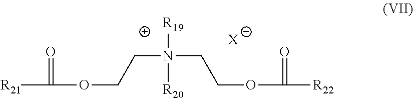

wherein
R₁₉, R₂₀, independently represent a C1-C6-Alkyl group and a C2-C6-hydroxyalkyl group,
R₂₁, R₂₂ independently represent a C7-C27-alkyl group, preferably an C10-C22-alkyl group and
X— stands for a physiologically compatible anion,
and/or the cosmetic product contains at least one cationic surfactant of the formula (VIII),

NR₂₃R₂₄R₂₅ (VIII)

wherein
R₂₃, R₂₄ independently represent an C1-C6-alkyl group, a C2-C6-alkenyl group or a C2-C6-hydroxyalkyl group,
R₂₅ represents a C8-C28-alkyl group, preferably a C10-C22-alkyl group and

The cationic surfactants of the formula (VIII) are amino derivatives, what are called pseudo quats. The organic radicals R₂₃, R₂₄ and R₂₅ are directly connected to the nitrogen atom in that case. In the acidic pH range, these are cationized i.e., the nitrogen atom is then protonized. The physiologically compatible counter-ions are then available as counter ions. Steamidopropyl dimethylamine is especially preferably offered with the cationic surfactants of the formula (VIII).

According to a preferred embodiment of the present invention, the cosmetic product contains a cationic surfactant as a further component. This preferably comprises a non-ionic surfactant selected from the group of

Alkyl glucamide, comprising a saturated or unsaturated, branched or unbranched C₆ to C₂₂, preferably C₁₀ to C₁₈, more preferably C₁₂ to C₁₆ alkyl group,

Alkyl fructoside, comprising a saturated or unsaturated, branched or unbranched C₆ to C₂₂, preferably C₁₀ to C₁₈, more preferably C₁₂ to C₁₆ alkyl group,

Alkyl glucoside, comprising a saturated or unsaturated, branched or unbranched C₆ to C₂₂, preferably C₁₀ to C₁₈, more preferably C₁₂ to C₁₆ alkyl group, and

Alkyl alcoholalkoxylate of the formula R₁₀(OR₁₁)ₘOH, in which R₁₀ represent a linear or branched C₆ to C₂₂, preferably C₁₀ to C₁₈, more preferably C₁₂ to C₁₆ alkyl group, R₁₁ represents a C₂ to C₄, preferably a C₂ alkyl group, and m represents about 1 to about 10, preferably about 2 to about 6, more preferably 2 to 6.

According to preferred embodiments of the present invention, the cosmetic product contains one or more anionic surfactants as a component, preferably selected from the group of

straight chained or branched, saturated or mono- or poly-unsaturated alkyl sulfonates with from about 8 to about 24, preferably from about 12 to about 22, more preferably from about 16 to about 18 C atoms,

linear alpha olefin sulfonates with from about 8 to about 24, preferably from about 12 to about 22, more preferably from about 16 to about 18 C atoms,

alkyl sulfates and alkyl polyglycol ether sulfates of the formula R₉—O—(CH₂—CH₂O)ₙ—SO₃X, in which R₉ preferably represents a straight chained or branched, saturated or mono- or poly-unsaturated alkyl or alkenyl radical with 8 from about to about 24, preferably from about 12 to about 22, more preferably from about 16 to about 18 carbon atoms, n stands for 0 or from about 1 to about 12, more preferably 2 to 4 and X for an alkali or alkali earth metal ion or for protonized tri-ethanol am or the ammonium ion.

straight chained or branched, saturated or mono- or poly-unsaturated alkyl carboxylic acids with from about 8 to about 24, preferably from about 12 to about 22, more preferably from about 16 to about 18 C atoms,

straight chained or branched, saturated or mono- or poly-unsaturated alkyl sulfonates with from about 8 to about 24, preferably from about 12 to about 22, more preferably from about 16 to about 18 C atoms,

Alkyl isethionate, whose alkyl group is selected from a branched or unbranched C₆ to C₂₂, preferably C₁₀ to C₁₈, more preferably C₁₂ to C₁₆ alkyl group, especially sodium cocoyl isethionate,

Alkyl glycoside carboxylic acids, whose alkyl group is selected from a branched or unbranched C₆ to C₂₂, preferably C₁₀ to C₁₈, more preferably C₁₂ to C₁₆ alkyl group,

Alkyl sulfosuccinates, whose two alkyl groups are selected from the same or different branched or unbranched C₂ to C₁₂, preferably C₄ to C₁₀, more preferably C₆ to C₈ alkyl groups,

Alkyl taurates, whose alkyl group is selected from a branched or unbranched C₆ to C₂₂, preferably C₁₀ to C₁₈, more preferably C₁₂ to C₁₆ alkyl group,

Alkylsarcosinates, whose alkyl group is selected from a branched or unbranched C₆ to C₂₂, preferably C₁₀ to C₁₈, more preferably C₁₂ to C₁₆ alkyl group,

Sulfonates of unsaturated fatty acids with from about 8 to about 24, preferably from about 12 to about 22, more preferably from about 16 to about 18 C atoms and from about 1 to about 6 double bonds,

Wherein the counter ion of the anionic surfactant is an alkali or earth alkali metal ion or a protonized triethanol amine or the ammonium ion.

Particularly preferred anionic surfactants are straight-chain or branched alkyl ether sulfates containing an alkyl radical with from about 8 to about 18 and in particular with from about 10 to about 16 C atoms and from about 1 to about 6 and in particular 2 to 4 ethylene oxide units,

specifically preferred, the surfactant mix of anionic and amphoteric/zwitterionic surfactants sodium lauryl ether sulfate (INCI: Sodium laureth sulfate) and specifically preferred sodium lauryl ether sulfate with 2 ethylene oxide units.

Amphoteric surfactants, which are also designated zwitterionic surfactants, are those surface-active compounds which carry at least one quaternary ammonium group and at least one —COO⁻— or —SO₃⁻ group in the molecule. Amphoteric/zwitterionic surfactants are also those surface-active compounds which, apart from a C₈-C₂₄ alkyl or acyl group, contain at least one free amino group and at least one —COOH— or —SO₃H— group in the molecule and are capable of forming internal salts.

According to a preferred embodiment of the present invention, the cosmetic product contains an amphoteric surfactant as a further component. The amphoteric surfactants in the cosmetic product are preferably selected from the group of

Alkyl betaine, comprising a saturated or unsaturated, branched or unbranched C₆ to C₂₂, preferably C₁₀ to C₁₈, more preferably C₁₂ to C₁₆ alkyl group,

Alkyl ampho diacetate or alkyl ampho diacetate, comprising a saturated or unsaturated, branched or unbranched C₆ to C₂₂, preferably C₁₀ to C₁₈, more preferably C₁₂ to C₁₆ alkyl group, with an alkali or earth alkali metal counter ion, and

Alkyl amide propyl betaine, comprising a saturated or unsaturated, branched or unbranched C₆ to C₂₂, preferably C₁₀ to C₁₈, more preferably C₁₂ to C₁₆ alkyl group,

The especially suitable amphoteric/zwitterionic surfactants also include the surfactants cocamidopropylbetaine and di-sodium cocoamphodiacetate known under the INCI designation.

The product for the treatment of a keratinous material can comprise especially an agent for cleaning a keratinous material, an agent for care of a keratinous material, an agent for care and cleaning of a keratinous material and/or an agent for temporary shaping of a keratinous material.

Other components of the hair treatment product shall be described hereafter, which can be contained in the products besides the above-described mandatory ingredients.

It can be preferable that the product for the treatment of a keratinous material further comprise from about 0.001 to about 20% by weight of at least one quaternary compound. This applies especially for products for care of a keratinous material and for products for care and cleaning of a keratinous material.

It is preferred that the at least one quaternary compound be selected from at least one of the groups of
i) the monoalkyl quats and/or
ii) the ester quats and/or
iii) the quaternary imidazoline of the formula (Tkat2).

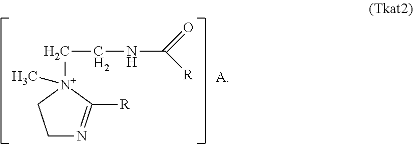

In which the radical R independently stands for a saturated or unsaturated, linear or branched hydrocarbon radical with a chain length from about 8 to about 30 carbon atoms and A stands for a physiologically compatible anion, and/or

iv) the amidoamines and/or cationized amidoamines and/or
v) poly(methacryloyloxyethyltrimethyl ammonium compounds) and/or
vi) quaternized cellulose derivatives, especially polyquaternium 10, polyquaternium-24, polyquaternium-27, polyquaternium-67, polyquaterium-72 and/or
vii) Cationic alkyl polyglycosides and/or
viii) Cationized honey and/or
ix) Cationic guar derivatives and/or
x) Chitosan and/or
xi) polymeric dimethyldiallylammonium salts and their copolymers with esters and amides of acrylic acid and methacrylic acid, mainly polyquaternium-7 and/or
xii) Copolymers of the vinyl pyrrolidone with quaternized derivatives of the dialkylaminoalkylacrylates and methacrylates, especially polyquaernium-11 and/or
xiii) vinylpyrrolidone-vinylimidazoliummethochloride-copolymers, especially polyquaternium-16 and/or
xiv) Quaternized polyvinyl alcohol and/or

### xv) Polyquaternium-74

and mixtures thereof.

It is especially preferred that the hair treatment product contain a cationic homopolymer, which falls under the INCI designation polyquaternium-37, as quaternary compounds.

It can be preferable that the product for the treatment of a keratinous material further comprise one solidifying compound, preferably selected from the group of waxes, synthetic polymers and mixtures thereof.

In order to cope with the different requirements for the product for the treatment of a keratinous material in the form of one agent for temporary shaping of a keratinous material (=Styling material), a multitude of synthetic polymers has already been developed as solidifying compounds, which can be used in the product for the treatment of a keratinous material. Waxes are used alternatively or additionally as solidifying compounds. Ideally, the polymers and/or waxes when used on the keratinous material leave a polymer film that lends a strong hold to the hairstyle on the one hand, is however sufficiently flexible on the other, so that it does not break on being stressed.

The synthetic polymers can be divided in cationic, anionic, anionic and amphoteric solidifying polymers.

Suitable synthetic polymers comprise, for example, polymers with the following INCI designation. Acrylamide/Ammonium Acrylate Copolymer, Acrylamides/DMAPA Acrylates/Methoxy PEG Methacrylate Copolymer, Acrylamidopropyltrimonium Chloride/Acrylamide Copolymer, Acrylamidopropyltrimonium Chloride/Acrylates Copolymer, Acrylates/Acetoacetoxyethyl Methacrylate Copolymer, Acrylates/Acrylamide Copolymer, Acrylates/Ammonium Methacrylate Copolymer, Acrylates/t-Butylacrylamide Copolymer, Acrylates Copolymer, Acrylates/C1-2 Succinates/Hydroxyacrylates Copolymer, Acrylates/Lauryl Acrylate/Stearyl Acrylate/Ethylamine Oxide Methacrylate Copolymer, Acrylates/Octylacrylamide Copolymer, Acrylates/Octylacrylamide/Diphenyl Amodimethicone Copolymer, Acrylates/Stearyl Acrylate/Ethylamine Oxide Methacrylate Copolymer, Acrylates/VA Copolymer, Acrylates/Hydroxy esters Acrylates Copolymer, Acrylates/VP Copolymer, Adipic Acid/Diethylenetriamine Copolymer, Adipic Acid/Dimethylaminohydroxypropyl Diethylenetriamine Copolymer, Adipic Acid/Epoxypropyl Diethylenetriamine Copolymer, Adipic Acid/Isophthalic Acid/Neopentyl Glycol/Trimethylolpropane Copolymer, Allyl Stearate/VA Copolymer, Aminoethylacrylate Phosphate/Acrylates Copolymer, Aminoethylpropanediol-Acrylates/Acrylamide Copolymer, Aminoethylpropanediol-AMPD-Acrylates/Diacetoneacrylamide Copolymer, Ammonium VA/Acrylates Copolymer, AMPD-Acrylates/Diacetoneacrylamide Copolymer, AMP-Acrylates/Allyl Methacrylate Copolymer, AMP-Acrylates/C1-18 Alkyl Acrylates/C1-8 Alkyl Acrylamide Copolymer, AMP-Acrylates/Diacetoneacrylamide Copolymer, AMP-Acrylates/Dimethylaminoethylmethacrylate Copolymer, Bacillus/Rice Bran Extract/Soybean Extract Ferment Filtrate, Bis-Butyloxyamodimethicone/PEG-60 Copolymer, Butyl Acrylate/Ethylhexyl Methacrylate Copolymer, Butyl Acrylate/Hydroxypropyl Dimethicone Acrylate Copolymer, Butylated PVP, Butyl Ester of Ethylene/MA Copolymer, Butyl Ester of PVM/MA Copolymer, Calcium/Sodium PVM/MA Copolymer, Corn Starch/Acrylamide/Sodium Acrylate Copolymer, Diethylene Glycolamine/Epichlorohydrin/Piperazine Copolymer, Dimethicone Cross polymer, Diphenyl Amodimethicone, Ethyl Ester of PVM/MA Copolymer, Hydrolyzed Wheat Protein/PVP Cross polymer, Isobutylene/Ethylmaleimide/Hydroxy ethylmaleimide Copolymer, Isobutylene/MA Copolymer, Isobutyl methacrylate/Bis-Hydroxypropyl Dimethicone Acrylate Copolymer, Isopropyl Ester of PVM/MA Copolymer, Lauryl Acrylate Cross polymer, Lauryl Methacrylate/Glycol Dimethacrylate Cross polymer, MEA-Sulfite, Methacrylic Acid/Sodium Acrylamidomethyl Propane Sulfonate Copolymer, Methacryloyl Ethyl Betaine/Acrylates Copolymer, Octylacrylamide/Acrylates/Butylaminoethyl Methacrylate Copolymer, PEG/PPG-25/25 Dimethicone/Acrylates Copolymer, PEG-8/SMDI Copolymer, Polyacrylamide, Polyacrylate-6, Polybeta-Alanine/Glutaric Acid Cross polymer, Polybutylene Terephthalate, Polyester-1, Polyethylacrylate, Polyethylene Terephthalate, Polymethacryloyl Ethyl Betaine, Polypentaerythrityl Terephthalate, Polyperfluoroperhydrophenanthrene, Polyquaternium-1, Polyquaternium-2, Polyquaternium-4, Polyquaternium-5, Polyquaternium-6, Polyquaternium-7, Polyquaternium-8, Polyquaternium-9, Polyquaternium-10, Polyquaternium-11, Polyquaternium-12, Polyquaternium-13, Polyquaternium-14, Polyquaternium-15, Polyquaternium-16, Polyquaternium-17, Polyquaternium-18, Polyquaternium-19, Polyquaternium-20, Polyquaternium-22, Polyquaternium-24, Polyquaternium-27, Polyquaternium-28, Polyquaternium-29, Polyquaternium-30, Polyquaternium-31, Polyquaternium-32, Polyquaternium-33, Polyquaternium-34, Polyquaternium-35, Polyquaternium-36, Polyquaternium-37, Polyquaternium-39, Polyquaternium-45, Polyquaternium-46, Polyquaternium-47, Polyquaternium-48, Polyquaternium-49, Polyquaternium-50, Polyquaternium-55, Polyquaternium-56, Polysilicone-9, Polyurethane-1, Polyurethane-6, Polyurethane-10, Polyvinyl Acetate, Polyvinyl Butyral, Polyvinylcaprolactam, Polyvinylformamide, Polyvinyl Imidazolinium Acetate, Polyvinyl Methyl Ether, Potassium Butyl Ester of PVM/MA Copolymer, Potassium Ethyl Ester of PVM/MA Copolymer, PPG-70 Polyglyceryl-10 Ether, PPG-12/SMDI Copolymer, PPG-51/SMDI Copolymer, PPG-10 Sorbitol, PVM/MA Copolymer, PVP, PVP/VA/Itaconic Acid Copolymer, PVP/VA/Vinyl Propionate Copolymer, Rhizobian Gum, Rosin Acrylate, Shellac, Sodium Butyl Ester of PVM/MA Copolymer, Sodium Ethyl Ester of PVM/MA Copolymer, Sodium Polyacrylate, Sterculia Urens Gum, Terephthalic Acid/Isophthalic Acid/Sodium Isophthalic Acid Sulfonate/Glycol Copolymer, Trimethylolpropane Triacrylate, Trimethylsiloxysilyl carbamoyl Pullulan, VA/Crotonates Copolymer, VA/Crotonates/Methacryloxybenzophenone-1 Copolymer, VA/Crotonates/Vinyl Neodecanoate Copolymer, VA/Crotonates/Vinyl Propionate Copolymer, VA/DBM Copolymer, VA/Vinyl Butyl Benzoate/Crotonates Copolymer, Vinylamine/Vinyl Alcohol Copolymer, Vinyl Caprolactam/VP/Dimethylaminoethyl Methacrylate Copolymer, VP/Acrylates/Lauryl Methacrylate Copolymer, VP/Dimethylaminoethyl methacrylate Copolymer, VP/DMAPA Acrylates Copolymer, VP/Hexadecene Copolymer, VP/VA Copolymer, VP/Vinyl Caprolactam/DMAPA Acrylates Copolymer, Yeast Palmitate and Styrene/VP Copolymer. Cellulose ethers, such as hydroxypropylcellulose, hydroxyethylcellulose and methylhydroxypropylcellulose are also suitable.

Also, Homopolyacryl acid (INCI: Carbomer), which is commercially available under the name Carbopol® in different versions, is suitable as a solidifying compound.

Preferably, the solidifying compound comprises a vinylpyrrolidone-containing polymer. Particularly preferably, the solidifying compound comprises a polymer selected from the group of polyvinylpyrrolidone (PVP), vinylpyrrolidone-vinylacetate-copolymer (VP/VA copolymer), vinyl caprolactam/VP/dimethylaminoethyl methacrylate copolymer (INCI), VP/DMAPA acrylates copolymer (INCI) and mixtures thereof.

A similarly preferred solidifying compound is octylacrylamide/acrylates/butylaminoethyl methacrylate copolymer (INCI), which is sold under the name “Amphomer®” by Akzo Nobel.

Accordingly, it is particularly preferable, that the solidifying compound comprises a synthetic polymer selected from the group of polyvinylpyrrolidone (PVP), vinylpyrrolidone-vinylacetate-copolymer (VP/VA copolymer), vinyl caprolactam/VP/dimethylaminoethyl methacrylate copolymer (INCI), VP/DMAPA acrylates copolymer (INCI), octylacrylamide/acrylates/butylaminoethyl methacrylate copolymer (INCI) and mixtures thereof.

The cosmetic composition can, additionally or alternatively to a synthetic polymer, contain at least one natural or synthetic wax, which has a melting point above 37° C., as solidifying compound.

As natural or synthetic waxes, solid paraffin or isoparaffins, vegetable waxes such candelila wax, carnauba wax, esparto grass wax, Japan wax, cork wax, sugar cane wax, ouricury wax, montan wax, sunflower wax, fruit waxes and animal waxes such as beeswaxes and other insect waxes, whale wax, shellac wax, wool wax and brushing grease, furthermore mineral waxes such as Ceresin and Ozokerite or the petrochemical waxes such as petrolatum, paraffin wax, microwaxes from polyethylene or polypropylene and polyethylene glycol waxes can be used. It can be advantageous to use hydrated or hardened waxes. Furthermore, chemically modified waxes, especially resin waxes, for example montan ester waxes, sasol waxes and hydrated jojoba waxes can also be used.

Furthermore, the triglycerides of saturated and unsaturated hydroxylized C 16-30 fatty acids, such as hardened triglyceride fats (hydrated palm oil, hydrated coconut oil, hydrated castor oil), glyceryl tribehenate or glyceryl tri-12 hydroxy stearate, are suitable in the cosmetic product.

The wax components can also be selected from the group of ester from saturated, unbranched alkane carboxylic acids of a chain length of from about 22 to about 24 C atoms and saturated, unbranched alcohols of a chain length of from about 22 to about 24 C atoms, if the wax components or the totality of the wax components are solid at room temperature. Silicone waxes, such as stearyltrimethylsilane/stearyl alcohol can also be eventually advantageous.

Natural, chemically modified and synthetic waxes can be used alone or in combination. This should, however, not include alkanes that are necessarily contained in the product as invented. Thus, even several waxes can be used. Moreover, a series of wax mixes, possibly in mixture with other additives, is also commercially available. Examples of usable mixtures are the ones available under the names “Special wax 7686 OE’ (a mixture of cetyl palmitate, bees wax, micro crystalline wax and polyethylene with a melting range of 73-75° C., manufacturer: Kahl & Co), Plywax® GP 200 (a mixture of stearyl alcohol and polyethylene glycol stearate with melting point of 47-51° C.; manufacturer: Croda) and “Weichceresin® FL 400” (a vaseline/vaseline oil/wax mixture with a melting point of 50-54° C.; manufacturer: Parafluid Mineralölgesellschaft).

The wax selected from Carnauba wax (INCI: Copernicia Cerifera Cera) bees wax (INCI: Beeswax), Petrolatum (INCI), micro crystalline wax and especially mixtures therefrom are preferred.

Preferred mixtures comprise the combination of Carnauba wax (INCI: Copernicia Cerifera Cera), Petrolatum and micro crystalline wax or the combination of bees wax (INCI: Beeswax) and Petrolatum.

The wax or the wax components should be solid at 25° C. and should melt in the range of >37° C.

The product for the treatment of a keratinous material contains the solidifying compound preferably in a total quantity of about 0.5 to about 50% by weight, preferably from about 1 to about 40% by weight, more preferred about 1.5 to about 30% by weight, still more preferred about 2 to about 25% by weight, based on the total weight of the cosmetic composition.

Other suitable ingredients comprise non-ionic polymers, anionic polymers, (other) cationic polymers, waxes, protein hydrolysates, amino acids, oligopetides, vitamins, pro-vitamins, vitamin precursors, betaines, biochinones, purine (derivatives), plant extracts, silicones, ester oils, UV light filters, structuring agents, thickening agents, electrolytes, pH adjusters, swelling agents, dyes, anti-dandruff agents, complexing agents, opacifiers, pearlizing agents, pigments, stabilizers, propellants, anti-oxidants, perfume oils and/or preservatives.

In the preferred embodiments 1 to 48, the preferred organic silicon compounds are mutually combined with the preferred alkane in an anhydrous carrier medium as invented.

| | Silane compound | Other ingredients |
|---|---|---|
| 1 | (3-Aminopropyl)trimethoxysilane | Branched or linear C14-C16 alkane |
| 2 | (3-Aminopropyl)triethoxysilan | Branched or linear C14-C16 alkane |
| 3 | (2-Aminoethyl)trimethoxysilane | Branched or linear C14-C16 alkane |
| 4 | (2-Aminoethyl)triethoxysilane | Branched or linear C14-C16 alkane |
| 5 | (3-Dimethylaminopropyl)trimethoxysilane | Branched or linear C14-C16 alkane |
| 6 | (3-Dimethylaminopropyl)triethoxy silane | Branched or linear C14-C16 alkane |
| 7 | (2-Dimethylaminoethyl)trimethoxy silane | Branched or linear C14-C16 alkane |
| 8 | (2-Dimethylaminoethyl)triethoxy silane | Branched or linear C14-C16 alkane |
| 9 | 3-(Trimethoxysilyl)-N-[3-(trimethoxysilyl) | Branched or linear C14-C16 alkane |
| | propyl]-1-propane amine | |
| 10 | 3-(Triethoxysilyl)-N-[3-(triethoxysilyl) | Branched or linear C14-C16 alkane |
| | propyl]-1-propane amine | |
| 11 | N-Methyl-3-(trimethoxysilyl)-N-[3- | Branched or linear C14-C16 alkane |
| | (trimethoxysilyl)propyl]-1-propane amine | |
| 12 | N-Methyl-3-(triethoxysilyl)-N-[3- | Branched or linear C14-C16 alkane |
| | (triethoxysilyl)propyl]-1-propane amine | |
| 13 | 2-[Bis[3-(trimethoxysilyl) propyl]amino]- | Branched or linear C14-C16 alkane |
| | ethanol | |
| 14 | 2-[Bis[3-(triethoxysilyl) propyl]amino]- | Branched or linear C14-C16 alkane |
| | ethanol | |
| 15 | 3-(Trimethoxysilyl)-N,N-bis[3- | Branched or linear C14-C16 alkane |
| | (trimethoxysilyl)propyl]-1-propane amine | |
| 16 | 3-(Triethoxysilyl)-N,N-bis[3- | Branched or linear C14-C16 alkane |
| | (triethoxysilyl)propyl]-1-propane amine | |
| 17 | N1,N1-Bis[3-(trimethoxysilyl)propyl]-1,2- | Branched or linear C14-C16 alkane |
| | ethane diamine | |
| 18 | N1,N1-Bis[3-(triethoxysilyl)propyl]-1,2- | Branched or linear C14-C16 alkane |
| | ethane diamine | |
| 19 | N,N-Bis[3-(trimethoxysilyl)propyl]-2- | Branched or linear C14-C16 alkane |
| | propene-1-amine | |
| 20 | N,N-Bis[3-(triethoxysilyl)propyl]-2-propene- | Branched or linear C14-C16 alkane |
| | 1-amine | |
| 21 | Methyltrimethoxysilane | Branched or linear C14-C16 alkane |
| 22 | Methyltriethoxysilane | Branched or linear C14-C16 alkane |
| 23 | Ethyltrimethoxy silane | Branched or linear C14-C16 alkane |
| 24 | Ethyltriethoxysilane | Branched or linear C14-C16 alkane |
| 25 | Propyltrimethoxysilane | Branched or linear C14-C16 alkane |
| 26 | Propyltriethoxysilane | Branched or linear C14-C16 alkane |
| 27 | Hexyltrimethoxysilane | Branched or linear C14-C16 alkane |
| 28 | Hexyltriethoxysilane | Branched or linear C14-C16 alkane |
| 29 | Octyltrimethoxysilane | Branched or linear C14-C16 alkane |
| 30 | Octyltriethoxysilane | Branched or linear C14-C16 alkane |
| 31 | Dodecyltrimethoxysilane | Branched or linear C14-C16 alkane |
| 32 | Dodecyltriethoxysilane | Branched or linear C14-C16 alkane |
| 33 | Octadecyltrimethoxysilane | Branched or linear C14-C16 alkane |
| 34 | Octadecyltriethoxysilane | Branched or linear C14-C16 alkane |
| 35 | (3-Aminopropyl)triethoxysilane + | Branched or linear C14-C16 alkane |
| | methyltrimethoxysilane | |
| 36 | (3-Aminopropyl)triethoxysilane + | Branched or linear C14-C16 alkane |
| | Methyltriethoxysilane | |
| 37 | (3-Aminopropyl)triethoxysilane + | Branched or linear C14-C16 alkane |
| | ethyltrimethoxysilane | |
| 38 | (3-Aminopropyl)triethoxysilane + | Branched or linear C14-C16 alkane |
| | ethyltriethoxysilane | |
| 39 | (3-Aminopropyl)triethoxysilane + | Branched or linear C14-C16 alkane |
| | propyltrimethoxysilane | |
| 40 | (3-Aminopropyl)triethoxysilane + | Branched or linear C14-C16 alkane |
| | propyltriethoxysilane | |
| 41 | (3-Aminopropyl)triethoxysilane + | Branched or linear C14-C16 alkane |
| | hexyltrimethoxysilane | |
| 42 | (3-Aminopropyl)triethoxysilane + | Branched or linear C14-C16 alkane |
| | hexyltriethoxysilane | |
| 43 | (3-Aminopropyl)triethoxysilane + | Branched or linear C14-C16 alkane |
| | octyltrimethoxysilane | |
| 44 | (3-Aminopropyl)triethoxysilane + | Branched or linear C14-C16 alkane |
| | octyltriethoxysilane | |
| 45 | (3-Aminopropyl)triethoxysilane + | Branched or linear C14-C16 alkane |
| | dodecyltrimethoxysilane | |
| 46 | (3-Aminopropyl)triethoxysilane + | Branched or linear C14-C16 alkane |
| | dodecyltriethoxysilane | |
| 47 | (3-Aminopropyl)triethoxysilane + | Branched or linear C14-C16 alkane |
| | octadecyltrimethoxysilane | |
| 48 | (3-Aminopropyl)triethoxysilane + | Branched or linear C14-C16 alkane |
| | octadecyltriethoxysilane | |

The product for the treatment of a keratinous material. Can already contain the active ingredients combination from at least one organic silicon compound and an alkane. In this embodiment, the product for the treatment of a keratinous material is already sold in ready-to-use form. In order to provide a formulation that is as stable as possible during storage, the product itself is preferably packaged with low or no water.

Alternatively, the at least one organic silicon compound is added to a base comprising all ingredients of the product for the treatment of a keratinous material with the exception of the at least one organic silicon compound, maximum 12 hours, preferably maximum 6 hours, more preferably maximum 3 hours, even more preferably maximum 1 hour before using the product for the treatment of a keratinous material.

Furthermore, alternatively, the organic silicon compound and the alkane are added to a cosmetic product just shortly before use, i.e., about 1 minute to about 12 hours, preferably from about 2 minutes to about 6 hours, especially preferably from about 1 minute to about 3 hours, most especially preferably from about 1 minute to about 1 hour.

In another alternative, the organic silicon compound is added to an aqueous solution, which is applied to the hair and in the second step, a cosmetic product, which already contains alkane, is applied on the hair.

The user can for example stir or shake an agent (α), which contains the organic silicon compound(s), first with an agent (β), which comprises the remaining ingredients of the product for the treatment of a keratinous material. The user can now apply this mixture of (α) and (β) to the keratinous materials—either directly after their production or after a short reaction time of about 1 minute to about 20 minutes. The agent (β) can contain water, especially water in a quantity >30% by weight, based on the total weight of the product for the treatment of keratinous materials.

Another object of the present application is the use of a cosmetic product for the treatment of a keratinous material as invented

for care of keratinous material,
for hydrophobizing the surface of keratinous material and/or
for improving the combability.

Regarding other preferred embodiments of the use, what was said about the cosmetic product is applicable mutatis mutandis.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. Cosmetic product for the treatment of a keratinous material, comprising
a) at least one organic silicon compound that comprises one to three silicon atoms and b) at least one branched or linear alkane, wherein the alkane is a C8-C30 alkane.

2. Cosmetic product for the treatment of a keratinous material as claimed in claim 1,
wherein
the at least one organic silicon compound, which comprises one to three silicon atoms, comprises a compound of the formula (I) and/or (II),
wherein in the organic silicon compound of formula (I)
R₁R₂N-L-Si(OR₃)ₐ(R₄)_{b} (I),
R₁, R₂ both represent a hydrogen atom,
L represents a linear, double-bonded C₁-C₆-alkylene group,
R₃, R₄ independently represent a methyl group or an ethyl group,
a stands for the number 3 and
b stands for the number 0, and
wherein in the organic silicon compound of formula (II)
(R₅O)_{c}(R₆)_{d}Si-(A)ₑ-[NR₇-(A′)]_{f}—[O-(A″)]_{g}—[NR₈-(A′″)]ₕ—Si(R₆′)_{d′}(OR₅′)_{c′} (II),
R₅, R₅′, R₅″, R₆, R₆′ and R₆″ independently represent a C₁-C₆ alkyl group,
A, A′, A″, A′″ and A″″ independently represent a linear or branched, double-bonded C₁-C₂₀-alkylene group,
R₇ and R₈ independently represent a hydrogen atom, a C₁-C₆-alkyl group, a hydroxy-C₁-C₆-alkyl group, a C₂-C₆-alkenyl group, an amino-C₁-C₆-alkyl group or a grouping of the formula (III)
-(A″″)-Si(R₆″)_{d}″(OR₅″)_{c}″ (III),
c stands for an integer from about 1 to about 3,
d stands for the integer 3-c,
c′ stands for an integer from about 1 to about 3,
d′ stands for the integer 3-c′,
c″ stands for an integer from about 1 to about 3,
d″ stands for the integer 3-c″,
e stands for 0 or 1,
f stands for 0 or 1,
g stands for 0 or 1,
h stands for 0 or 1,
with the proviso that at least one of the radicals from e, f, g and h is different from 0.

3. Cosmetic product for the treatment of a keratinous material as claimed in claim 2,
wherein
the product for the treatment of a keratinous material comprises at least one organic silicon compound of the formula (I), which is selected from the group of
(3-Aminopropyl)trimethoxysilane
(3-Aminopropyl)triethoxysilane
(2-Aminoethyl)trimethoxysilane
(2-Aminoethyl)triethoxysilane
(3-Dimethylaminopropyl)trimethoxysilane
(3-Dimethylaminopropyl)triethoxysilane
(2-dimethylaminoethyl)trimethoxysilane and/or
(2-Dimethylaminoethyl)triethoxysilane.

4. Cosmetic product for the treatment of a keratinous material as claimed in claim 1,
wherein
the product for the treatment of a keratinous material comprises at least one organic silicon compound of the formula (II), which is selected from the group of
3-(trimethoxysilyl)-N-[3-(trimethoxysilyl)propyl]-1-propanamine
3-(Triethoxysilyl)-N-[3-(triethoxysilyl)propyl]-1-propanamine
N-methyl-3-(trimethoxysilyl)-N-[3-(trimethoxysilyl)propyl]-1-propanamine
N-Methyl-3-(triethoxysilyl)-N-[3-(triethoxysilyl)propyl]-1-propanamine
2-[Bis[3-(trimethoxysilyl)propyl]amino]-ethanol
2-[Bis[3-(triethoxysilyl)propyl]amino]ethanol
3-(Trimethoxysilyl)-N,N-bis[3-(trimethoxysilyl)propyl]-1-propanamine
3-(Triethoxysilyl)-N,N-bis[3-(triethoxysilyl)propyl]-1-propanamine
N1,N1-Bis[3-(trimethoxysilyl)propyl]-1,2-ethanediamine,
N1,N1-Bis[3-(triethoxysilyl)propyl]-1,2-ethanediamine,
N,N-Bis[3-(trimethoxysilyl) propyl]-2-propene-1-amine and/or
N,N-Bis[3-(triethoxysilyl)propyl]-2-propene-1-amine.

5. Cosmetic product for the treatment of a keratinous material as claimed in claim 1, wherein the cosmetic product comprises the organic silicon compound in a quantity of from about 0.01 to about 10% by.

6. The cosmetic product for the treatment of a keratinous material as claimed in claim 1, wherein the cosmetic product further comprises a component c), which is a skin moisturizer selected from the group of glycerin, urea, hyaluronic acid, silanol ester of the hyaluronic acid, panthenol, taurine, ceramides, phytosteroles, aloe vera extracts, creatine, creatinine, sodium hyaluronate, polysaccharides, biosaccharides gum-1, cucumber extracts, butylene glycol, propylene glycol, methyl propanediol, ethylhexylglycerin, sorbitol, amino acids, lactic acid, lactates, and/or ethylhexyloxyglycerin.

7. Cosmetic product for the treatment of a keratinous material as claimed in claim 1, wherein the cosmetic product comprises two structurally mutually different surfactants.

8. Cosmetic product for the treatment of a keratinous material as claimed in claim 1, wherein the pH value of the cosmetic product is from about 1 to about 9.

9. Cosmetic product for the treatment of a keratinous material as claimed in claim 1, wherein the quantity of branched or linear alkane in the cosmetic product is from about 0.1 to about 30% by weight.

10. Cosmetic product for the treatment of a keratinous material as claimed in claim 1, wherein the product for the treatment of a keratinous material comprises at least one organic silicon compound of the formula (IV)
R9Si(OR₁₀)ₖ(R₁₁)ₘ (IV).

11. Cosmetic product for the treatment of a keratinous material as claimed in claim 1, wherein the cosmetic product for the treatment of a keratinous material comprises at least two structurally mutually different organic silicon compounds, each of which comprises one to three silicon atoms.

12. Cosmetic product for the treatment of a keratinous material as claimed in claim 1, wherein the cosmetic product for the treatment of a keratinous material comprises:
from about 0.5 to about 5% by weight of at least one first organic silicon compound selected from the group of (3-Aminopropyl)trimethoxysilane, (3-Aminopropyl)triethoxysilane, (2-Aminoethyl)trimethoxysilane, (2-Aminoethyl)triethoxysilane, (3-Dimethylaminopropyl)trimethoxysilane, (3-Dimethylaminopropyl)triethoxysilane (2-Dimethylaminoethyl)trimethoxysilane and (2-Dimethylaminoethyl)triethoxysilane, and
from about 3.2 to about 7% by weight of at least one second organic silicon compound selected from the group of methyltrimethoxysilane, methyltriethoxysilane, ethyltrimethoxysilane, ethyltriethoxysilane, propyltrimethoxysilane, propyltriethoxysilane, hexyltrimethoxysilane, hexyltriethoxysilane, octyltrimethoxysilane, octyltriethoxysilane, dodecyltrimethoxysilane, dodecyltriethoxysilane, octadecyltrimethoxysilane and octadecyltriethoxysilane.

13. (canceled)

14. Cosmetic product for the treatment of a keratinous material as claimed in claim 1, wherein the alkane is a C12-C18 alkane.

15. Cosmetic product for the treatment of a keratinous material as claimed in claim 1, wherein the cosmetic product comprises the organic silicon compound in a quantity of from about 0.1 to about 7% by weight, based on the total weight of the cosmetic product.

16. Cosmetic product for the treatment of a keratinous material as claimed in claim 1, wherein the organic silicon compound is (3-aminopropyl)triethoxysilane.

17. Cosmetic product for the treatment of a keratinous material as claimed in claim 1, wherein the cosmetic product comprises the organic silicon compound in a quantity of from about 0.1 to 7% by weight, based on the total weight of the cosmetic product.

18. Cosmetic product for the treatment of a keratinous material as claimed in claim 16, wherein the organic silicon compound is (3-aminopropyl)triethoxysilane.

19. The cosmetic product for the treatment of a keratinous material as claimed in claim 6, wherein component c) is chosen from glycine, glycine soy, histidine, tyrosine or tryptophan.

20. Cosmetic product for the treatment of a keratinous material as claimed in claim 10, wherein the at least one organic silicon compound of the formula (IV) is selected from the group of
Methyltrimethoxysilane
Methyltriethoxysilane
Ethyltrimethoxysilane
Ethyltriethoxysilane
Propyltrimethoxysilane
Propyltriethoxysilane
Hexyltrimethoxysilane
Hexyltriethoxysilane
Octyltrimethoxysilane
Octyltriethoxysilane
Dodecyltrimethoxysilane
Dodecyltriethoxysilane.
Octadecyltrimethoxysilane and/or
Octadecyltriethoxysilane.
